SEQUENCE LISTING (15 sequences)

SEQ ID NO: 1 - DNA, 22 bp, synthetic construct
  source 1..22: mol_type = other DNA
gaattcyactattttggaggac

SEQ ID NO: 2 - DNA, 19 bp, synthetic construct
  source 1..19: mol_type = other DNA
ttgacaagcaattgcctgg

SEQ ID NO: 3 - DNA, 23 bp, synthetic construct
  source 1..23: mol_type = other DNA
ccaggaggcacactagaagatac

SEQ ID NO: 4 - DNA, 20 bp, synthetic construct
  source 1..20: mol_type = other DNA
tgacactgtgcctcaatcct

SEQ ID NO: 5 - DNA, 22 bp, unidentified
  source 1..22: mol_type = other DNA
acaactgggagtcagaggtaac

SEQ ID NO: 6 - DNA, 24 bp, synthetic construct
  source 1..24: mol_type = other DNA
aggtgaagcacgcatacctgtgcc

SEQ ID NO: 7 - DNA, 22 bp, synthetic construct
  source 1..22: mol_type = other DNA
ggctgaaagagaaagaggtatc

SEQ ID NO: 8 - DNA, 21 bp, synthetic construct
  source 1..21: mol_type = other DNA
gatgaaatctctgtgacctgg

SEQ ID NO: 9 - DNA, 23 bp, synthetic construct
  source 1..23: mol_type = other DNA
ttcgaaactccaaaataccacgt

SEQ ID NO: 10 - DNA, 21 bp, synthetic construct
  source 1..21: mol_type = other DNA
agtcagtgatttcggctcgtt

SEQ ID NO: 11 - DNA, 21 bp, synthetic construct
  source 1..21: mol_type = other DNA
gcctgccaagccgataaagga

SEQ ID NO: 12 - DNA, 20 bp, synthetic construct
  source 1..20: mol_type = other DNA
agctgccaaacagccttcag

SEQ ID NO: 13 - DNA, 22 bp, synthetic construct
  source 1..22: mol_type = other DNA
gtgcaaaaccttactatgttgt

SEQ ID NO: 14 - DNA, 20 bp, synthetic construct
  source 1..20: mol_type = other DNA
gaacgtccatatgttccaaa

SEQ ID NO: 15 - DNA, 24 bp, synthetic construct
  source 1..24: mol_type = other DNA
tgggtgtaattgctcgttgtgtag

(19) United States
(12) Patent Application Publication
(10) Pub. No.: US 2025/0387104 A1
(43) Pub. Date: Dec. 25, 2025

(54) SYSTEM FOR TESTING FEMALE REPRODUCTIVE HEALTH AND DIAGNOSTIC METHOD

(71) Applicant: Jiangsu BioPerfectus Technologies Co., Ltd, Taizhou City (CN)

(72) Inventors: Guohui ZHOU, Taizhou City (CN); Rong ZHANG, Taizhou City (CN); Yuwan GUO, Taizhou City (CN); Yonghong CHEN, Taizhou City (CN); Ying WU, Taizhou City (CN); Tao LI, Taizhou City (CN)

(21) Appl. No.: 19/242,840

(22) Filed: Jun. 18, 2025

Related U.S. Application Data
(60) Provisional application No. 63/669,982, filed on Jul. 11, 2024.

(30) Foreign Application Priority Data
Jun. 25, 2024 (CN) 202410877546.9

(51) Int. Cl. A61B 10/00; A61B 5/00; A61F 13/472; C12Q 1/48; C12Q 1/6806; C12Q 1/6809; C12Q 1/6851; C12Q 1/686; C12Q 1/689

(52) U.S. Cl. CPC A61B 10/0045; A61B 5/4294; A61F 13/472; C12Q 1/48; C12Q 1/6806; C12Q 1/6809; C12Q 1/6851; C12Q 1/686; C12Q 1/689; C12Y 207/07; A61B 2010/0074

(57) ABSTRACT

The present disclosure provides a system for testing a health status of a female vagina, including: a collecting element for collecting a female vaginal secretion, wherein the collecting element covers an external surface of a vagina instead of entering the vagina, and is capable of absorbing the vaginal secretion; and a testing element for testing an analyte in the collected vaginal secretion or a property of the analyte to assess a health status of the vagina based on a test result obtained by the testing element.

### CROSS-REFERENCE TO RELATED APPLICATIONS

The present disclosure claims priority to Chinese Prior Application No. 2024108775469, filed on Jun. 25, 2024, and U.S. Provisional Application No. 63/669,982, filed on Jul. 11, 2024, the entire contents of which are incorporated herein as part of the present disclosure, including but not limited to the specification, abstract, claims, and drawings.

### REFERENCE TO AN ELECTRONIC SEQUENCE LISTING

The content of the electronic sequence listing (1347US2559.xml; Size: 14,135 bytes; and Date of Creation: Jun. 17, 2025) is herein incorporated by reference in its entirety.

### BACKGROUND OF THE INVENTION

### Field of the Invention

The present disclosure relates to a device for collecting a liquid sample and a test device, and in particular to, a device for testing an analyte in a liquid sample in the field of rapid diagnosis, such as a system or method for testing an analyte in urine or a female vaginal secretion or a property of the analyte.

### Description of the Related Art

The following background art description is merely an introduction to some common background knowledge and shall not impose any limitation on the present invention.

In healthy women, the vagina possesses a natural defense function against pathogen invasion due to the characteristics of anatomical tissues. For example, the closure of the vaginal introitus, keratinization of epithelial cells, and the maintenance of vaginal acidity at pH 4-5 inhibit the proliferation of alkaline-adapted pathogens such as anaerobic bacteria. When the natural defense function of the vagina is damaged, pathogens can easily invade, leading to vaginal inflammation.

The vaginal inflammation is a common and frequently-occurring disease in women. Besides causing symptoms of vulvovaginal discomfort in patients, it can also lead to a series of serious complications related to reproductive health, endangering female reproductive health. Common vaginal inflammations include bacterial vaginosis (BV), aerobic vaginitis (AV), trichomoniasis, vulvovaginal candidiasis (VVC), and the like.

The female vaginal microecosystem is a component of the human microecosystem, and consists of vaginal microbial floras, an endocrine regulatory system, a vaginal anatomical structure, and a local immune system. The vaginal microbial floras are diverse, featuring mutual symbiosis and antagonism among species. Influenced by various internal and external factors, they participate in forming a structurally complex microecosystem. In a normal vaginal microbial flora, Lactobacillus serves as dominant bacteria, which may coexist with a small number of other miscellaneous bacteria. Vaginal lactobacilli play a key role in maintaining the normal vaginal flora. Glycogen in the vaginal epithelium is broken down into lactic acid by lactobacilli, creating a weakly acidic local environment in the vagina. The following situations potentially disrupt the balance of the vaginal ecosystem: (1) Sex hormones: for example, decreased estrogen levels before and after menstruation can lead to an increase in vaginal pH, favoring the growth of anaerobic bacteria. (2) Some spermicidal contraceptives: have toxic effects on lactobacilli. (3) Drugs: many drugs, such as broad-spectrum antibiotics, can kill or inhibit lactobacilli, thereby affecting the vaginal environment. (4) Infection: for example, promiscuity can disrupt the original vaginal flora, leading to dysbiosis. When the vaginal microecological balance is disrupted, changes characterized by abnormal vaginal flora and abnormal vaginal pH can occur. This represents a trend that can reduce the vagina's resistance to pathogenic microorganisms, leading to secondary infections. In addition, when infected with some communicable diseases, such as acquired immune deficiency syndrome and human papillomavirus (HPV), symptoms may also appear in the female vagina.

Whether testing properties of female vaginal secretions, e.g., pH or the quantity of an analyte, for example, testing oxidase, hydrogen peroxide, leukocyte esterase, sialidase, β-glucuronidase, acetylglucosaminidase, or a pH value, sampling of vaginal secretions is required before analysis can be performed.

In traditional sampling, for example, the sampler disclosed in WO2015190738A1 has a tube with an open rear and a closed front end, providing an internal space where a finger can be inserted into the female vagina for sampling of a secretion inside the vagina. A self-vaginal sampler includes a collecting part arranged on a sheath at a closed end of a tube and configured to collect a cervical sample, commonly referred to as a “vaginal swab”, e.g., a cotton swab. Such similar samplers require inserting part of the structure deep into the vagina, which can potentially cause damage to vaginal tissues. Furthermore, traditional sampling methods require testers to perform the operation themselves, which can cause discomfort and is not user-friendly. Particularly, self-sampling is more cumbersome, and the sampling is insufficient. Additionally, during sampling, most testers do not insert the sampler deep into the vagina but instead lightly swab the external surface of the vagina. The traditional sampling methods described above not only cause discomfort of testers during sampling but can also lead to the defect of missed detection due to inaccurate sampling.

To solve the technical problems of the aforementioned traditional products, improvements are necessary to provide alternative solutions to overcome the shortcomings of existing traditional techniques.

### BRIEF SUMMARY OF THE INVENTION

To overcome multiple defects caused by similar “vaginal swab” sampling in traditional vaginal secretion sampling methods, the present disclosure employs a modified feminine sanitary pad for vaginal secretion sampling, which not only achieves the convenience of the sampling operation but also solves the problem of missed detection.

Therefore, on the one hand, the present disclosure provides a system for testing a female vaginal health status, including: a collecting element for collecting a female vaginal secretion, wherein the collecting element covers an external surface of a vagina instead of entering the vagina, so as to collect the vaginal secretion, and is configured to absorb the vaginal secretion; and a testing element for testing an analyte in the collected vaginal secretion or a property of the analyte to assess a health status of the vagina based on a test result obtained by the testing element so as to assess a female reproductive status. The system overcomes the defects of traditional sampling, a sheet-like flexible material is directly used for covering an external surface of a vagina; over time, the sheet-like absorbent material can absorb a vaginal secretion; and then the analyte in the vaginal secretion is tested.

In some specific embodiments, the collecting element includes a sheet-like absorbent material that covers the external surface of the vagina so as to continuously absorb the vaginal secretion, such that the secretion in the vagina is retained on the absorbent material, and the secretion absorbed by the absorbent material is for testing the analyte so as to obtain the presence or absence of the analyte or the quantity present thereof. Based on the quantity present, the health status of the female vagina is judged. In some embodiments, the absorbent material includes a flexible material that is absorbent, such as a commonly used feminine sanitary pad, a tampon, toilet paper, or a urine absorbent sheet. In some embodiments, the sheet-like absorbent material covers the female vaginal surface for a specific time without detachment. In some specific embodiments, the time includes 2-20 hours, preferably 5-8 hours. In some specific embodiments, the sheet-like absorbent material has a back surface and a front surface, and an adhesive layer is disposed on the back surface. By disposing the adhesive layer on the back surface of the absorbent material, the adhesive layer is fixed to the skin on the external surface of the vagina without detachment, and the adhesive layer covers the vaginal surface, such that it can be ensured that the absorbent material can absorb the vaginal secretion.

In some embodiments, to reduce subsequent interference with testing of different analytes in the absorbed sample, improvements are made to the materials and properties of traditional absorbent layers as well as other auxiliary layers, thereby reducing subsequent interference with testing of analytes.

In some embodiments, the absorbent material includes two layers of absorbent material, including a first layer and a second layer. The back surface of the first layer includes the adhesive layer. The first layer of absorbent material is disposed on the adhesive layer. A pH test paper layer is disposed on the first layer of absorbent material and covered by the second layer. The first layer of absorbent material includes a dense surface and a loose surface. Adhesive is coated on the dense surface, while a pH test paper is disposed on the loose surface. The dense surface can directly cover the skin around the vagina for adhesion around the vagina, such that the dense surface is in direct contact with the vaginal orifice. When the secretion permeates the adhesive layer and passes through the first layer to flow to the pH test paper layer, the pH value of the secretion can be tested for preliminary judgment of a vaginal inflammation status. In some embodiments, when a nucleic acid of a virus is tested, the sample used is from a sample absorbed by the second layer. In some embodiments, an area of the second layer for absorbing the sample is not in direct contact with an area of the pH filter paper. In some embodiments, the first layer includes a back surface and a front surface. The pore size of the back surface is smaller than that of the front surface. Furthermore, the back surface has an adhesive layer, which further reduces the pore size of the back surface and makes water permeation difficult, such that other large impurities in the secretions, such as proteins, polysaccharides, or red blood cells, can be filtered or blocked, thereby avoiding interference. In some embodiments, the front surface of the first layer includes a large-pore-size material, e.g., flexible materials such as cotton or large-pore-size filter papers. In this way, through the adhesion of the adhesive layer on the back surface and its role as a barrier for macromolecules, it plays a role in filtering, such that the secretion is filtered through the adhesive layer, flows to the front surface, and is then in contact with the pH test paper layer for testing the pH value of the secretion. In some embodiments, in this way, a sample area for sampling may be the front surface of the first layer or the second layer. In some preferred embodiments, sampling from the first layer is most suitable. In some embodiments, when the filter paper layer is located between the first layer and the second layer, the sampling area is an area of the first layer and the second layer not in contact with the filter paper, such as areas at the two ends of the filter paper.

Certainly, in some embodiments, the adhesive layer can adhere to underwear, and then the second layer directly covers the external surface of the vagina by wearing the underwear, such that the vaginal secretion is directly absorbed by the second layer, and the excess secretion flows to the pH test paper to test the pH value. If there is still a remaining sample, it flows onto the first layer. When sampling, sampling from the first layer enables more accurate testing of the quantity of the analyte. This is likely because the second layer filters out proteins, polysaccharides, or other red blood cells, thereby reducing interference during the extraction of viral nucleic acids from cells and also reducing interfering substances in nucleic acid amplification. Crucially, it is found that most of the virus-containing cells remain on the first layer.

In some embodiments, the analyte is for nucleic acid amplification testing, such as testing for viral or bacterial nucleic acids. In some embodiments, the testing is viral nucleic acid testing. In some embodiments, the viruses include: human papillomavirus (HPV), including high-risk types (e.g., HPV 16, 18) that are associated with cervical cancer, and low-risk types (e.g., HPV 6, 11) that may cause genital warts; herpes simplex virus (HSV), e.g., HSV-2 that commonly causes genital herpes, leading to painful vesicles and ulcers; human immunodeficiency virus (HIV) that is present in vaginal secretions and transmissible through sexual contact; and cytomegalovirus (CMV) that is typically asymptomatic, but may be transmitted through mother-to-child transmission or sexual contact.

In some embodiments, the second layer may cover the first layer by means of easy tearing off. When sampling is required, the second layer is torn directly off from the first layer, and then sampling is carried out from the sample on the second layer, for example, nucleic acid extraction is carried out after the sampling. In some embodiments, two ends of the second layer adhere to two ends of the first layer using an adhesive layer. In some embodiments, the second layer is provided with multiple areas that are connected by easily-torn lines or easily-torn openings. When sampling, a part of a sample area is removed directly from the second layer and then subsequent nucleic acid extraction is proceeded. In some embodiments, the sheet-like absorbent material is provided with different areas, and the areas are capable of being detachably removed from a collecting element. For example, the sheet-like material is a non-absorbent substrate, such as a flexible plastic or a flexible film. One or more layers of absorbent material adhere to the flexible film. The absorbent material can be removed or torn from the flexible film, such that the absorbent materials can absorb the secretion. Certainly, the absorbent material may have easily-torn lines, such that the absorbent material can be separated into multiple small pieces that contain the vaginal secretion. In some specific embodiments, the different areas of the sheet-like absorbent material are capable of being directly cut off to obtain partial samples. The vaginal secretion typically has inherent coloration while the absorbent material may be colorless or white, such that the area that contains the secretion naturally develops color, indicating that the area absorbs the secretion. The absorbent material containing the analyte is directly removed for subsequent testing.

In some specific embodiments, the absorbent material for the secretion can be dissolved, eluted, subjected to nucleic acid extraction, or direct nucleic acid amplification.

In some specific embodiments, the testing element includes a nucleic acid testing element, an immunoassay element, or a biochemical testing element. In some specific embodiments, the nucleic acid testing element is used for testing a quantity or presence or absence of a target nucleic acid in the secretion, and preferably, the nucleic acid includes a virus, a bacterium, or a fungus, more preferably, the virus. In some specific embodiments, the virus is human papillomavirus (HPV) or a sexually transmitted disease (STD). In some specific embodiments, the STD includes syphilis, condyloma acuminata, acquired immune deficiency syndrome, or gonorrhea. In some specific embodiments, the nucleic acid testing element includes reagents necessary for polymerase chain reaction (PCR), isothermal amplification, and nucleic acid amplification. In some specific embodiments, biochemical testing or immunoassay is for testing a viral antigen, or oxidase, hydrogen peroxide, leukocyte esterase, sialidase, β-glucuronidase, acetylglucosaminidase, or a pH value in the analyte.

On the other hand, the present disclosure provides a method for testing a female vaginal health status, including: covering an external surface of a vagina with a sheet-like element including an absorbent material for a specific time for absorbing a vaginal secretion; and then removing a part of the absorbent material including the secretion from the sheet-like element for testing an analyte in the secretion.

In some specific embodiments, the sheet-like absorbent material is a feminine sanitary pad. In some specific embodiments, the removing includes cutting off (the sheet-like material itself is an absorbent material) or tearing off (the absorbent material adhering to the sheet-like element). In some specific embodiments, the specific time includes 2-20 hours, preferably 5-8 hours. In some specific embodiments, the covering is self-applied by a patient on her own external vaginal surface; and the removing a part of the absorbent material including the secretion is performed by laboratory personnel or the patient herself.

In some specific embodiments, the analyte in the secretion can be tested using nucleic acid amplification, immunoassay, and biological testing. In some specific embodiments, the analyte includes viruses such as human papillomavirus (HPV), syphilis, condyloma acuminata, acquired immune deficiency syndrome, or gonorrhea, or oxidase, hydrogen peroxide, leukocyte esterase, sialidase, β-glucuronidase, acetylglucosaminidase, or a pH value. In some specific embodiments, whether the vagina is healthy or has a certain disease is judged from the test result. In some specific embodiments, the disease includes vaginal inflammation, bacterial inflammation, or a certain virus or a specific viral load.

In the above sampling methods, if the vagina has a certain disease or inflammation, it naturally secretes more secretions, making sampling easier and collection more friendly. For example, the following types of inflammation all involve increased secretions: Vaginitis: vaginitis is a common gynecological disease including many types, such as bacterial vaginitis, trichomonal vaginitis, and vulvovaginal candidiasis. The vaginitis can cause symptoms in women including pruritus vulvae, increased vaginal secretions, abnormal color and shape of secretions, and local erosion. Pelvic inflammation: the tissue anatomy structure of the pelvis is relatively special and easily infected by bacteria, causing inflammation. This is a common gynecological disease that can cause symptoms such as high fever, chills, increased leucorrhea, and hypogastralgia. Cervicitis: cervicitis is a chronic inflammation that generally occurs at any age stage. It can cause congestion and edema of the cervix. This disease can cause abnormal vaginal secretion in women, along with symptoms such as frequent urination, urgent urination, and lower abdominal pain. Uterine myoma: uterine myoma is a common benign tumor of female reproductive organs, usually appearing in women aged 30 to 50. Women with uterine myoma may feel a mass in the lower abdomen and experience symptoms such as soreness and pain in the waist and back, increased leucorrhea, and a dragging sensation and fullness in the lower abdomen.

### BENEFICIAL EFFECT

The system and method mentioned above enable user-friendly and convenient collection of vaginal secretions, particularly suitable for at-home self-sampling.

### BRIEF DESCRIPTION OF THE DRAWINGS

FIG. 1A shows a structural exploded view of a sanitary pad in a specific embodiment of the present disclosure.

FIG. 1B shows a structural top view of a first layer in a specific embodiment of the present disclosure.

FIG. 1C shows a sectional structural schematic diagram of adhering a loose layer to a front surface of a first layer in another specific embodiment of the present disclosure.

FIG. 2 shows a structural schematic diagram of a flexible sanitary pad in a specific embodiment of the present disclosure.

FIG. 3 shows a structural schematic diagram of a flexible sanitary pad in a specific embodiment of the present disclosure, where a second layer is protected by a protective layer.

FIG. 4 shows an operational schematic diagram of tearing off a part of a second layer from the sanitary pad of the present disclosure in a specific embodiment of the present disclosure.

FIG. 5 shows a structural schematic diagram of a second layer with a part that is tearable after sample collection using the sanitary pad of the present disclosure in a specific embodiment of the present disclosure.

FIG. 6 shows a structural schematic diagram of a sanitary pad in a specific embodiment of the present disclosure, where multiple tearable areas are disposed on a second layer.

FIG. 7 shows a structural schematic diagram of a sanitary pad in a specific embodiment of the present disclosure and a schematic diagram of an operational process of tearing off a tearable area from a second layer.

FIG. 8 shows a structural schematic diagram of a sanitary pad in a specific embodiment of the present disclosure.

FIG. 9 shows a schematic structural exploded view of a sanitary pad in a specific embodiment of the present disclosure.

FIG. 10 shows a structural schematic diagram of a sanitary pad in a specific embodiment of the present disclosure.

FIG. 11 shows a structural schematic diagram of a sanitary pad in a specific embodiment of the present disclosure.

### DETAILED DESCRIPTION OF THE INVENTION

The structures involved in the present disclosure or the technical terms used are further explained below. Unless otherwise specified, they shall be understood and explained according to the general terms commonly used in the art.

### Testing

Testing means to assay or test the presence or absence of a substance or material, including but not limited to, a chemical substance, an organic compound, an inorganic compound, a metabolite, a drug or a drug metabolite, an organic tissue or a metabolite of an organic tissue, a nucleic acid, a protein, or a polymer. In addition, testing means testing the quantity of a substance or material. Further, assay also means immunoassay, chemical assay, enzymatic assay, and the like.

### Sample

Samples for the test device of the present disclosure include biological fluids (e.g., case fluids or clinical samples). Liquid samples or fluid samples can be derived from solid or semi-solid samples, including feces, biological tissues, and food samples. The solid or semi-solid samples can be converted into liquid samples using any suitable method, such as mixing, mashing, maceration, incubation, dissolution, or enzymatic digestion of the solid samples in an appropriate solution (e.g., water, phosphate solution, or other buffer solutions). Certainly, in some examples, the sample may be a vaginal secretion. A flexible sanitary pad is used for collecting the vaginal secretion. The flexible sanitary pad contains an absorbent material that covers the vaginal surface to absorb the vaginal secretion.

### Vaginal Secretion

The general composition of a vaginal secretion includes mucus, cells, bacteria, and the like. Specifically, it may include cervical mucus, exfoliated cells from the vaginal wall, lactobacilli, and potentially white blood cells. The secretion also contains some electrolytes or enzymes like lysozyme, which may aid in bactericidal activity. pH is also a crucial factor; it is normally acidic, approximately 4-4.5, primarily maintained by lactic acid produced by lactobacilli. Whether the number of exfoliated epithelial cells and white blood cells is within the normal range is significant; for example, a small number of white blood cells is normal, but an increase in white blood cells may indicate inflammation. Certainly, the vaginal secretion also includes, for example, water, proteins, and polysaccharides. The vaginal secretion may also include electrolytes like sodium, potassium, and chloride ions. Furthermore, mucopolysaccharides and glycoproteins in cervical mucus are important components with relatively high specific gravity. The vaginal secretion is a complex mixture of various components, and its normal component reflects the health status of the reproductive system. Primary sources of the secretion include: cervical mucus: secreted by the cervical gland, and influenced by estrogens (increases during ovulation, being egg-white-like); vaginal wall transudate: interstitial fluid and exfoliated cells from the vaginal mucosa; greater vestibular gland (Bartholin's gland): secretes mucus to lubricate the vaginal introitus during sexual arousal; and endometrial fluid: small amounts mixed in during the menstrual cycle.

Specific components of the vaginal secretion: water (>90%): maintains fluidity of the secretion; electrolytes: sodium, potassium, chloride ions that maintain osmotic pressure; organic substances: glycoproteins and mucopolysaccharides (e.g., mucin in cervical mucus) that form gel structures to trap pathogens; lactic acid: produced by lactobacilli breaking down glycogen, maintains pH 3.8-4.5; enzymes (lysozyme, catalase): antibacterial action; short-chain fatty acids: assist in inhibiting microbial overgrowth; cellular components: exfoliated squamous epithelial cells: normal metabolic products; white blood cells (small number, <5 per high-power field): immune surveillance; and red blood cells: present only during menstruation or abnormal bleeding; microorganisms: include dominant floras: Lactobacillus (e.g., Lactobacillus crispatus, Lactobacillus gasseri): >70%, produce H₂O₂ and bacteriocins; commensal flora: Streptococcus, Staphylococcus (coagulase-negative), Gardnerella (low load), Anaerobes (Prevotella, Mobiluncus), and Mycoplasma (e.g., Mycoplasma hominis). Dynamic balance of microbial floras: the proportions of flora are regulated by hormones, pH, and immune factors. Main functions of these dominant floras: self-cleaning function: an acidic environment inhibits pathogens (e.g., Candida albicans proliferates easily at pH >4.5); mucus barrier: physically blocks ascending infection by pathogens; immune signaling: cytokines (e.g., IL-1β, defensins) in the secretion participate in local immune responses. When abnormal components appear in the secretion, it suggests an unhealthy female reproductive system. The abnormal components include: pathogens: Candida hyphae, Trichomonas, and Clue cells (clusters of Gardnerella). Biochemical changes: pH >4.5, positive amine test (bacterial vaginitis).

### Analyte

For example, analytes tested by the present disclosure include, but are not limited to: creatinine, bilirubin, nitrites, proteins (non-specific), hormones (e.g., human chorionic gonadotropin, progesterone, and follicle-stimulating hormone), blood, leucocytes, glucose, heavy metals or toxins, bacterial substances (e.g., proteins or carbohydrates against specific bacteria such as Escherichia coli O157:H7, Staphylococcus, Salmonella, Clostridium, Campylobacter, L. monocytogenes, Vibrio, or Bacillus cereus), and substances in a urine sample related to physiological characteristics, such as pH and specific gravity. Any other clinical chemistry analysis can be performed using lateral flow testing in combination with the device of the present disclosure. Viruses in the vaginal secretion can also be tested, such as HIV, HPV, or any other viruses, bacteria, as well as other biochemical indicators like secretion pH. For example, oxidase, hydrogen peroxide, leukocyte esterase, sialidase, β-glucuronidase, and acetylglucosaminidase in the secretion can sometimes distinguish bacterial inflammation, for example, tests of indicators such as hydrogen peroxide, leukocyte esterase, sialidase, and pH value.

### Reproductive Tract Infection

Reproductive tract infections are common gynecological diseases in women and represent a global social and public health issue. Common reproductive tract infections mainly include vaginitis (bacterial, trichomonal, candidal, and aerobic vaginitis), sexually transmitted diseases (STDs), and cervical diseases. The primary cause of reproductive tract infections is an invasion of the reproductive tract by various microorganisms such as bacteria, viruses, mycoplasmas, fungi, and trichomonads. In the normal vaginal flora, a reduction or absence of lactobacilli (Lactobacillus crispatus, Lactobacillus gasseri, Lactobacillus jensenii, and Lactobacillus iners) as dominant bacteria weakens their inhibitory effect on other microorganisms, allowing pathogenic microorganisms to proliferate excessively, leading to vaginitis. Bacterial vaginosis, aerobic vaginitis, trichomonal vaginitis, and vulvovaginal candidiasis account for over 70%-90% of vaginitis cases. Among vaginitis patients, bacterial vaginosis accounts for approximately 36%-60%, aerobic vaginitis accounts for approximately 9.4%-23.7%, trichomonal vaginitis accounts for approximately 0.3%-20%, and vulvovaginal candidiasis accounts for approximately 15%-40%.

Primary microorganisms causing bacterial vaginosis include Gardnerella vaginalis, Atopobium vaginae, Prevotella, Mobiluncus, Bacteroides, Megasphaera, bacterial vaginosis-associated bacterium 2 (BVAB2), Ureaplasma urealyticum, Mycoplasma hominis, etc. Primary microorganisms causing aerobic vaginitis include Group A Streptococcus, Group B Streptococcus, Escherichia coli, Staphylococcus aureus, Enterococcus faecalis, Streptococcus anginosus, and Klebsiella pneumoniae. The microorganism causing trichomonal vaginitis is Trichomonas vaginalis. Vulvovaginal candidiasis is a reproductive tract disease caused by infection with Candida. The predominant causative pathogen is Candida albicans, while non-albicans Candida such as Candida glabrata, Candida tropicalis, and Candida krusei account for a minority.

STDs are a category of widely prevalent diseases. Gonorrhea is among the most common, primarily caused by the microorganism Neisseria gonorrhoeae. In cases of non-gonococcal urethritis, the common microorganism is Chlamydia trachomatis. Mycoplasma genitalium can also cause non-gonococcal urethritis. Treponema pallidum is the microorganism that causes syphilis, and more than 95% of syphilis infections result from sexual contact transmission. Herpes simplex virus types 1 and 2 can lead to vesicular infections in the genital and perianal areas, causing sexually transmitted diseases. There are over 200 subtypes of HPV that can infect humans, among which more than 40 can cause human diseases. It is primarily transmitted through contact. Persistent HPV infection can cause hyperplastic lesions of the human reproductive tract mucosa and skin, clinically manifesting as various types of epithelial warts, as well as malignant tumors such as cervical cancer, anal cancer, and vaginal cancer. Based on the risk levels of HPV, WHO classifies HPV into 14 high-risk types: HPV 16, 18, 31, 33, 35, 39, 45, 51, 52, 56, 58, 59, 66, and 68; 4 probable high-risk types: HPV 26, 53, 73, and 82; with the remainder classified as low-risk types. Persistent infection with high-risk HPV is the primary cause of cervical cancer and its precancerous lesions.

The microorganisms mentioned above may cause infection individually, or multiple microorganisms may cause mixed infections. Mixed infections of reproductive tract microorganisms are often associated with the presence of a complex reproductive tract environment. Compared with single-microorganism infections, the diagnosis and treatment of mixed infections are more difficult. The various microorganisms mentioned above that can cause reproductive tract infections, such as bacteria, viruses, mycoplasmas, fungi, and trichomonas, are all present in the vaginal secretion. Therefore, usually, to comprehensively detect the pathogenic causes of vaginal inflammation, especially the types of microorganisms, it is necessary to collect the vaginal secretion for testing multiple microorganisms. However, collecting with a vaginal swab a limited number of times cannot fully test for all pathogenic bacteria in a single attempt. This is primarily due to the limited sample collected and the very short duration of collection.

Additionally, the vaginal secretion can also be analyzed for cancer markers or exfoliated cancer cells. These markers are present in the vaginal secretion, and by detecting cancer or tumor marker molecules, the presence or absence or likelihood of cancer in a female reproductive system can also be directly detected.

### Vaginal Health Status or Disease

The “vaginal health status or disease” defined in the present disclosure refers to the reproductive health status or disease that can be indicated by the results of vaginal secretion tests. This disease is not for confirmation, but it can at least indicate a possibility. The common categories are as follows:

One category is inflammatory diseases, such as the following inflammatory diseases: vaginitis: a common gynecological disease including many types like bacterial vaginitis, trichomonal vaginitis, and vulvovaginal candidiasis. The vaginitis can cause symptoms in women including pruritus vulvae, increased vaginal secretions, abnormal color and shape of secretions, and local erosion. Pelvic inflammation: the tissue anatomy structure of the pelvis is relatively special and easily infected by bacteria, causing inflammation. This is a common gynecological disease that can cause symptoms such as high fever, chills, increased leucorrhea, and hypogastralgia. Cervicitis: cervicitis is a chronic inflammation that generally occurs at any age stage. It can cause congestion and edema of the cervix. This disease can cause abnormal vaginal secretion in women, along with symptoms such as frequent urination, urgent urination, and lower abdominal pain. Uterine myoma: uterine myoma is a common benign tumor of female reproductive organs, usually appearing in women aged 30 to 50. Women with uterine myoma may feel a mass in the lower abdomen and experience symptoms such as soreness and pain in the waist and back, increased leucorrhea, and a dragging sensation and fullness in the lower abdomen, as well as some markers.

Indicators for testing inflammation include pH value, oxidase, hydrogen peroxide, leukocyte esterase, sialidase, β-glucuronidase, and acetylglucosaminidase, which can sometimes distinguish bacterial inflammation, for example, tests of indicators such as hydrogen peroxide, leukocyte esterase, sialidase, and pH value. The indicators mentioned above can be tested using a biochemical method, i.e., using chemical reagents containing chromogenic substrates. When these substances react with the chemical reagents, a color change on a testing pad indicates the quantity or content of these analytes. This is a preliminary reaction of inflammation. However, if inflammation cannot be tested using a chemical method, it does not mean inflammation is absent. Testing for various microorganisms at this stage can enable early prediction, thereby achieving the effects of early prevention and treatment.

The other category is infectious diseases, such as virus infections like HPV and STDs, e.g., HPV, syphilis, condyloma acuminata, acquired immune deficiency syndrome, or gonorrhea. Methods for testing such viruses may be immunoassays or nucleic acid tests. Immunoassays can use lateral flow test strips or any other immunological method. Nucleic acid tests include any nucleic acid amplification method, such as temperature-cycling PCR, isothermal nucleic acid amplification techniques, or tests for target nucleic acids utilizing gene editing technologies. Certainly, reagents necessary for nucleic acid tests are also included, such as buffers for nucleic acid extraction, and enzymes, metal ions, primers, and probes for nucleic acid amplification, as well as nucleic acid extraction reagents. These reagents for nucleic acid tests (primers for nucleic acid extraction, nucleic acid amplification, and target nucleic acids) can be purchased commercially, or commercially available kits from the applicant of the present disclosure can be used for testing.

### Downstream and Upstream

Downstream or upstream is divided according to the direction of fluid flow. Generally, liquid or fluid flows from an upstream area to a downstream area. The downstream area receives the liquid from the upstream area. The liquid can also flow to the downstream area along the upstream area. Herein, downstream or upstream is generally divided according to the direction of fluid flow. For example, on some materials where capillary force drives fluid flow, the liquid can overcome gravity and flow in the direction opposite to gravity; in this case, upstream and downstream are still divided by the direction of fluid flow. For example, in the test device of the present disclosure, when a liquid sample, e.g., a liquid sample from a sample chamber, such as urine or saliva sample from a test subject, enters the testing chamber bottom, the fluid can flow from the sample application area to the label area, then to the testing area, e.g., a test result area and a test result control area. The testing area may be a polyester fiber film. The liquid diversion element may be a glass fiber, a polyester sheet, or a polyester film.

### Gas Communication or Liquid Communication

Gas communication or liquid communication means that liquid or gas can flow from one place to another. In the flow process, the liquid or the gas may pass through some physical structures that play a guiding role. The “passing through some physical structures” generally means the liquid passes through the surface of these structures or through internal spaces within them to flow to another place passively or actively. Passive flow is induced by external forces, such as capillary action or air pressure. The flow herein can also occur due to the liquid's or gas's own action (gravity or pressure), or it can be passive flow. Fluid flow under air pressure can be forward or reverse, or the air pressure can cause the fluid to flow from one place to another. The communication herein does not mean that liquid or gas is necessarily present. It merely indicates a connection relationship or state between two objects in some cases. If liquid is present, it can flow from one object to another. Herein, it means a state in which two objects are connected. Conversely, if two objects are not in fluid or gas communication, liquid in or on one object cannot flow into or onto the other object, and this is a non-communication, non-liquid communication, or non-gas communication state.

### Testing Element

The “testing element” herein refers to an element capable of testing whether a fluid sample or fluid specimen (a liquid sample or liquid specimen) contains an analyte of interest. Such testing may be based on any technical principle, including immunological, chemical, electrical, optical, molecular, nucleic acid-based, physical, etc. The testing element can be a lateral flow test strip that can test multiple analytes. Certainly, other suitable testing elements can also be used in the present disclosure.

Various testing elements can be combined and used in the present disclosure. One form is a test strip. Test strips for analyzing analytes in samples (e.g., drugs or metabolites indicating physiological condition) can take various forms, such as immunoassays or chemical analysis. Test strips can utilize non-competitive or competitive assay formats. A test strip generally includes an absorbent material with a sample application area, a reagent area, and a testing area. A fluid or liquid sample is applied to the sample application area and flows to the reagent area via capillary action. In the reagent area, if the analyte is present, the sample combines with a reagent. The sample then continues to flow to the testing area. Other reagents, such as molecules specifically binding to the analyte, are immobilized in the testing area. These reagents react with the analyte (if present) in the sample and bind to the analyte in this area, or bind to a reagent from the reagent area. A label for displaying a test signal is present in the reagent area or a separate label area.

In a typical non-competitive assay format, a signal is generated if the analyte is present in the sample and no signal is generated if the analyte is absent. In a competitive assay format, a signal is generated if the analyte is absent in the sample and no signal is generated if the analyte is present.

The testing elements may be test strips, made of absorbent or non-absorbent materials. A test strip can include various materials for liquid sample transport. One material of the test strip can cover another. For example, filter paper covers a nitrocellulose (NC) membrane. One area of the test strip may be made of one or more materials, while another area is made of one or more different materials. The test strip can adhere to a support or rigid surface to enhance the strength of holding the test strip.

The analyte is tested through a signal generating system. For example, by using one or more enzymes that specifically react with the analyte, and by using the above method of immobilizing a specific binding substance on the test strip, a combination of one or more signal generating systems is immobilized in the analyte testing area of the test strip. Signal-generating substances may be in the application area, the reagent area, or the testing area, or throughout the test strip; they can saturate one or more materials of the test strip. A signal substance-containing solution is applied to the surface of the test strip or one or more materials of the test strip are immersed in the signal substance-containing solution. The test strip with the applied signal substance-containing solution is dried.

Generally, commonly used test strips are NC membrane test strips, that is, the testing area includes an NC membrane on which specific binding molecules are immobilized to display a test result. Other membranes like cellulose acetate membrane or nylon membrane can also be used. For example, test strips and devices including test strips, as described in the following patents: U.S. Pat. Nos. 4,857,453; 5,073,484; 5,119,831; 5,185,127; 5,275,785; 5,416,000; 5,504,013; 5,602,040; 5,622,871; 5,654,162; 5,656,503; 5,686,315; 5,766,961; 5,770,460; 5,916,815; 5,976,895; 6,248,598; 6,140,136; 6,187,269; 6,187,598; 6,228,660; 6,235,241; 6,306,642; 6,352,862; 6,372,515; 6,379,620, and 6,403,383. The test strips and the similar devices with test strips disclosed in the above patents can be applied to the testing element or test device of the present disclosure for testing the analyte, for example, testing an analyte in a sample.

The test strips applied in the present disclosure can be conventional lateral flow test strips. Their specific structure and test principles are well-known to those of ordinary skill in the art. A common test strip includes a sample collection area or a sample application area, a label area, and a testing area. The sample collection area includes a sample receiving pad. The label area includes a label pad. An absorbent area may include an absorbent pad. The testing area includes necessary chemical substances capable of detecting the presence or absence of the analyte, such as immunoreagents or enzyme chemical reagents. The test strip has the sample application area. Generally, commonly used test strips are NC membrane test strips, that is, the testing area includes an NC membrane on which specific binding molecules are immobilized to display a test result. Other membranes like cellulose acetate membrane or nylon membrane can also be used. Certainly, a test result control area can also be included downstream of the testing area. The control area and the testing area generally appear as horizontal lines, namely, a test line or a control line. Such test strips are conventional. Certainly, other types of test strips for testing using capillary action can also be used. Furthermore, test strips generally contain dry chemical reagents, such as immobilized antibodies or other reagents. When the test strip contacts a liquid, the liquid flows along the test strip via capillary action. With the flow, the dry reagents are dissolved in the liquid and then react in the next area to perform the necessary test. The liquid flow is primarily driven by capillary action Herein, All these types can be applied in the test device of the present disclosure, disposed within the testing chamber to be in contact with the liquid sample, or used for testing the presence or absence or quantity of the analyte in the liquid sample entering the testing chamber. Lateral flow can be used for testing tumor markers and microorganisms such as bacteria, fungi, and viruses in the vaginal secretion. During testing, pre-treatment of the vaginal secretion to remove interfering substances is necessary before testing antigens on viruses.

Certainly, the nucleic acid testing elements are generally used by a test device or system based on nucleic acid amplification. Any nucleic acid amplification technology can be used in the present disclosure. For example, PCR amplification technology, e.g., digital PCR technology, real-time fluorescence PCR technology, isothermal amplification technology, or gene editing technology, for nucleic acid tests. The technologies mentioned above all enable nucleic acid testing and assays. The nucleic acid testing elements generally include nucleic acid extraction kits, nucleic acid amplification reagents, primers, and probes, as well as fluorescent dyes.

### Sanitary Pad Sampling

The flexible sampling element described herein refers to an element that can directly cover the external vaginal surface, e.g., the surrounding skin outside the female vagina, to receive the vaginal secretion. The element receiving the secretion generally includes an absorbent material, such as filter paper, sponge, foam, non-woven fabrics, or feminine sanitary pads. In some preferred embodiments, a modified feminine sanitary pad is used as one method of the present disclosure to collect the vaginal secretion for testing the analyte in the secretion.

This differs significantly from a traditional method for secretion collection by a vaginal swab (e.g., cotton swab). The traditional method for vaginal secretion collection by a vaginal swab is that the vaginal swab needs to be inserted into the vagina for sampling or swab the external vaginal surface to absorb the sample, which has the following defects or limitations: one is limited sampling sites: although instructions may guide operators to perform multi-point sampling, they often sample a single-point. Although the vaginal secretion is absorbed in single-point sampling, it may not contain the analyte, leading to missed detection. In addition, swab sampling obtains a small sample volume (50 microliters or 1 milliliter), which is often inadequate. For example, when aiming to simultaneously test multiple analytes or various microorganisms in the secretion, this frequently results in missed detection. Another is an operational inconvenience: when collecting vaginal analytes (e.g., 10, 20, 30, 40, 50, or more types), when aiming for a larger volume of vaginal secretion, multiple samplings are necessary to ensure sufficient functional sample quantity. Multiple samplings are complex and time-consuming and cause significant discomfort or reluctance in the individuals being sampled. Third, swab sampling requires self-collection by the individual being tested. Incorrect operation, such as improper insertion depth or improper vaginal surface application, can cause injury to vaginal tissue. Fourth, sampling by a vaginal swab, e.g., cotton swab, is completed within 2-3 minutes. Single sampling cannot obtain a sufficient sample for all indicators. When vaginal microorganisms need to be tested in a single attempt, usually, when aiming to test at least 20, 30, 40, or 50 types by single sampling, this cannot be completed by single swab sampling within a short time.

Therefore, the present disclosure uses improved common feminine sanitary pads for sampling, overcoming the defects of traditional swab sampling. In some embodiments, on the one hand, such a sanitary pad is absorbent and can absorb and retain the vaginal secretion. In addition, the sampling method is the same as the routine use of the sanitary pad during the menstrual cycle, greatly enhancing operational comfort. Finally, the improved sanitary pad is used for sampling, allowing for the collection of all vaginal secretions in a single attempt, and can be used repeatedly. Whether testing for a single analyte or multiple analytes, it can be completed by single sampling. Furthermore, the sampling duration can last several hours, generally corresponding to the duration of sanitary pad wearing. It may be more than one hour, e.g., 2-8 hours. The sanitary pad is generally worn before bedtime at night and removed in the morning, such that the sample can be collected for a longer time. In this way, the sanitary pad can absorb not only the secretion from the vaginal surface but also the liquid secreted from deeper vaginal regions. Consequently, the sanitary pad can absorb and retain floras and exfoliated cells from the surface, as well as internal floras and exfoliated cells, and multiple microorganisms can be tested in a single attempt without missed detection.

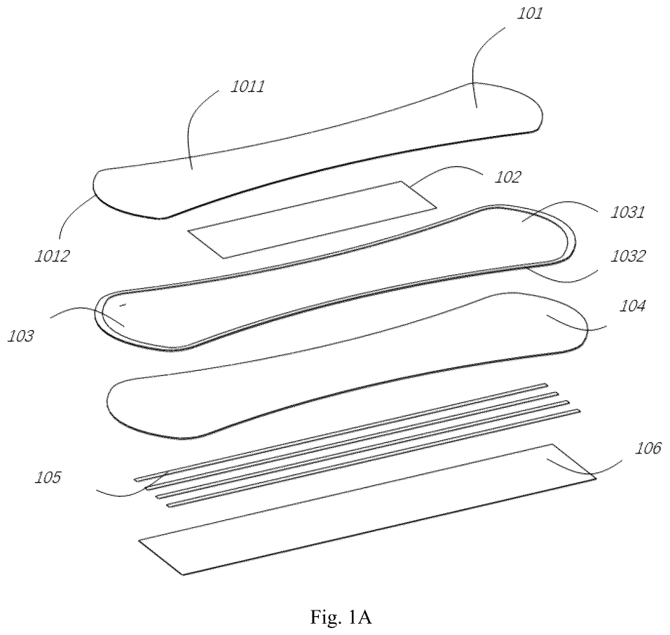

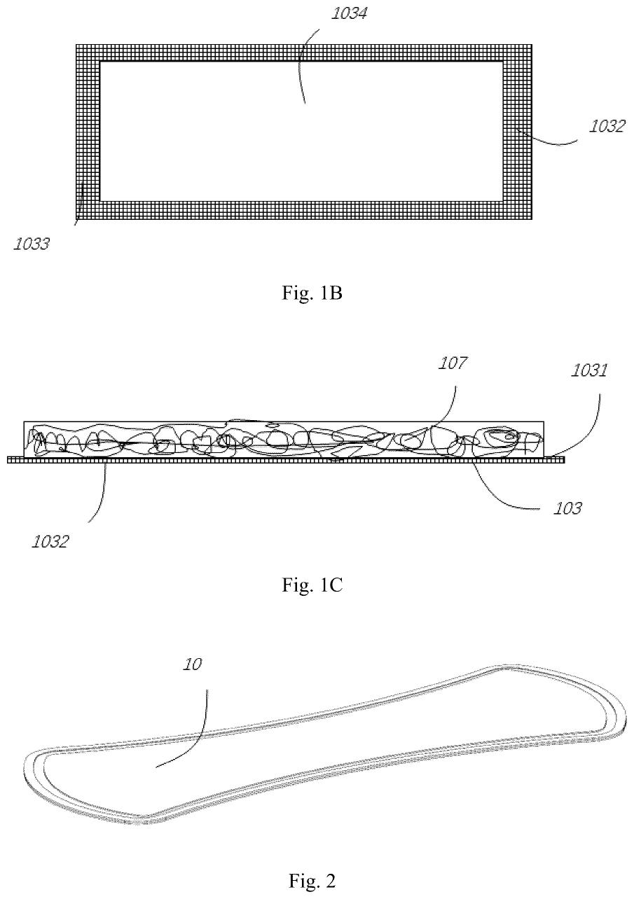

In some embodiments, as shown in FIGS. 1A-1C, the improved sanitary pad includes a first-layer absorbent material 103 and a second-layer absorbent material 101. A pH test paper 102 is disposed between the first-layer absorbent material 103 and the second-layer absorbent material 101 and used for testing a pH value of a vaginal secretion. First, the vaginal inflammation status is judged by the color change of the pH test paper. However, this provides only a preliminary indication and does not indicate the absence of viral infection. Certainly, the pH test paper may be excluded. In some embodiments, the first-layer absorbent material 103 includes a front surface 1031 and a back surface 1032. The back surface 1032 is dense, e.g., shaped like a dense mesh. The front surface 1031 is a loose surface 107. An adhesive layer 104 is coated on the dense surface 1032 for adhering to the skin around the vagina or to the underwear. In some embodiments, the second layer 101 also has a dense surface 1011 (back surface) and a loose surface 1012 (front surface). The loose surface 1031 of the first layer 103 and the loose surface 1012 of the second layer 101 face each other, and a pH test paper is disposed between the loose surfaces of the two layers. The terms “dense” and “loose” are relative. Generally, the dense surface is of a small-pore-size mesh-like structure similar to textiles (FIG. 1B); the loose surface is like a dense surface covered with loose materials, or materials with large pore size, such as a thin layer of cotton, flocked fibers, or plant fibers (as shown in FIG. 1C). Alternatively, the first layer 103 itself is a dense layer 103. Short fibers, cotton, etc., are fixed to the front surface 1031 to form a loose surface, while a pH filter paper is disposed on the loose layer. Alternatively, the second layer 101 itself is a dense layer, and short fibers, e.g., flocked fibers, are fixed to the front surface 1012 of the second layer, also forming a loose surface. When the loose surface of the first layer and the loose surface of the second layer are combined face-to-face, a loose layer is formed between them. Furthermore, both the first layer and the second layer are dense layers, and a loose layer 107 is disposed between them, thereby forming a sanitary pad collector for collecting the vaginal secretion. The fixation method may be adhesive adhering that adheres loose cotton or fibers to the front surface 1031 of the first layer 103 or the front surface 1012 of the second layer 101. Alternatively, the first layer and the second layer adhere around their periphery, and then a loose layer approximately 2-3 millimeters thick is filled between the two layers.

Therefore, in some embodiments, one surface of the loose layer 107 is made of materials such as cotton or flocking. Herein, “dense” and “loose” are relative states. The dense layer features a small pore size, a compact texture, and a thickness of approximately 0.2-1 millimeter, while the loose surface is provided with fixed cotton or fluff, featuring a large pore size, a fluffy texture, and a thickness of approximately 2-3 millimeters. When the sanitary pad adheres and is fixed to the underwear via the adhesive layer 104, the dense surface 1011 of the second layer 101 covers the external surface of the vagina, such as the skin surrounding the vagina, such that the vaginal secretion is first in contact with the dense surface 1011 of the second layer 101. The dense layer can filter out red blood cells, proteins, polysaccharides, or large-particle substances (curd-like substances). Therefore, the dense surface has small pores and can fundamentally block the passage of red blood cells, proteins, polysaccharides, or large curd-like particles, and other substances, such as mixed liquids of water and cells (exfoliated cells), as well as microorganisms in the water, can pass through the dense surface 1011 and flow onto the loose layer 107, and is in contact with the pH test paper on the loose layer to measure a pH value. The remainder is retained in the loose layer for subsequent analyte tests, such as nucleic acid extraction for nucleic acid amplification or marker tests. In some embodiments, the first layer is a dense layer 103, and the second layer is also a dense layer 101. A loose layer 107, e.g., cotton, flocking, or short fibers approximately 2-5 mm thick, is disposed between the two layers. The back surface 1032 of the first layer has an adhesive layer 104, by which the sanitary pad adheres to the underwear for sampling or collecting the vaginal secretion. The sample is collected from the loose layer 107 between the first layer 103 and the second layer 101 for testing. In some embodiments, the sample from the loose layer 107 undergoes nucleic acid extraction from cells, such that viral infections can be effectively tested. The sanitary pad is particularly suitable for nucleic acid amplification testing, effectively and accurately reflecting the true health status of the vagina without missed detection.

The “curd-like substance” in the present disclosure is a characteristic feature of VVC, primarily caused by the overgrowth of Candida albicans or other Candida species. The curd-like substance is a component of the vaginal secretion, which appears white or milk-white with a thick, curd-like, or lumpy texture resembling curd or cottage cheese, often adhering to the vaginal wall or vulva.

In some embodiments, the adhesive layer 104 can adhere directly to the skin around the periphery of the vagina, such that the vaginal secretion is first in contact with the adhesive layer 104. Although the adhesive layer is a layer of adhesive, it allows the liquid to pass through the dense back surface 1031 and enter the loose layer 107 to be absorbed. It can be understood that the adhesive layer does not cover the entire back surface 1032 of the first layer. Instead, a small area of adhesive can be applied to the two ends of the back surface 1032 of the first layer, or the adhesive can be applied around the periphery 1033 of the back surface 1032 of the first layer, while no adhesive is applied to a central area 1034. The loose layer 107 is disposed at the central area. For example, as shown in FIG. 3B, the adhesive layer 1033 is applied around the periphery of the back surface 1032 of the first layer 103, while the central area 1034 contains no adhesive. This can also achieve adhesion to the skin around the vagina. When implemented this way, the vaginal secretion is first in contact with the partial area 1034 of the back surface 1032 of the first layer. Large-particle substances are first blocked and filtered out. The vaginal secretion then passes through the dense layer and enters the loose layer 107 (as shown in FIG. 1C), where the liquid is absorbed and serves as the sample for subsequent extraction. Certainly, the sample can also be treated subsequently directly using the dense layer or the dense surface 1032 together with the loose layer 107. However, the sample treatment process is relatively complex. First, large-particle substances need to be removed, such as red blood cells, proteins, or polysaccharides, before nucleic acid extraction. Performing direct nucleic acid extraction without this step would increase interfering substances, leading to inaccurate test results. Using the sample from the loose layer allows for direct nucleic acid extraction followed by nucleic acid amplification.

In some embodiments, the first layer or the second layer is formed by multiple absorbent strips arranged, or the second layer is also formed by multiple absorbent materials. The dense surfaces of the multiple absorbent materials form the dense surface of the first layer or the dense surface of the second layer, and the loose layer adheres to the first layer or the second layer. The multiple absorbent strips are connected by easily-torn lines 1018. During sampling, one absorbent strip 1013 is torn directly from the first layer 101. A specific area of the loose layer is then cut off from this absorbent strip for sample treatment, such as nucleic acid extraction. Certainly, each absorbent reagent strip may also be formed by multiple small absorbent blocks connected by easily-torn lines, such that a small block can be directly torn off for sample treatment, with each small block to which a loose layer adheres.

Therefore, in some embodiments, the sanitary pad for collecting the vaginal secretion includes a first layer 103, a second layer 101, and a third layer 107. The first layer 103 is a dense layer, and the second layer 101 is also a dense layer. The loose third layer 107 is disposed between the first layer and the second layer. The third layer adheres to the first layer or the second layer. A pH test paper is disposed between the third layer and the first layer, or between the third layer and the second layer. For example, when the loose third layer adheres to the first layer, the pH test paper is disposed between the second layer and the third layer. When the third layer 107 adheres to the second layer 101, the pH test paper is disposed between the third layer and the first layer. In some embodiments, the loose third layer 107 adheres to the front surface 1012 of the second layer 101. The second layer includes small blocks 1014, 1015, and 1016 formed by multiple easily-torn lines. During sampling, a small block, e.g., 1015, is torn directly from the second layer 101. This small block includes not only a dense surface but also the loose layer adhering to its front surface. In this case, the loose layer can be directly removed for sample treatment, or the dense layer and the loose layer can be communicated for sample treatment.

In some embodiments, a release liner 105 is further disposed on the adhesive layer 104, and a protective layer 106 is disposed on the release liner. In this way, the sanitary pad is protected by the release liner. During use, the release liner 105 is torn off directly from the adhesive layer 104, for example, from the back surface 1042 of the adhesive layer 104, the adhesive layer is protected, such that the adhesive layer can adhere to the underwear or directly onto the skin around the vagina. The protective layer 106 is disposed to prevent the release liner from separation.

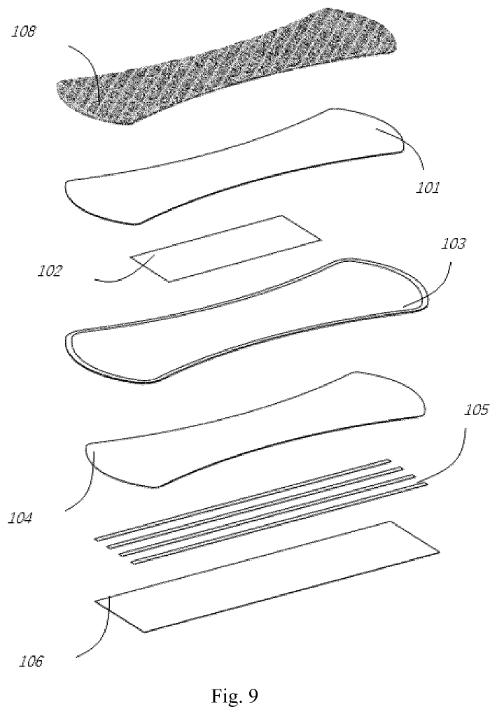

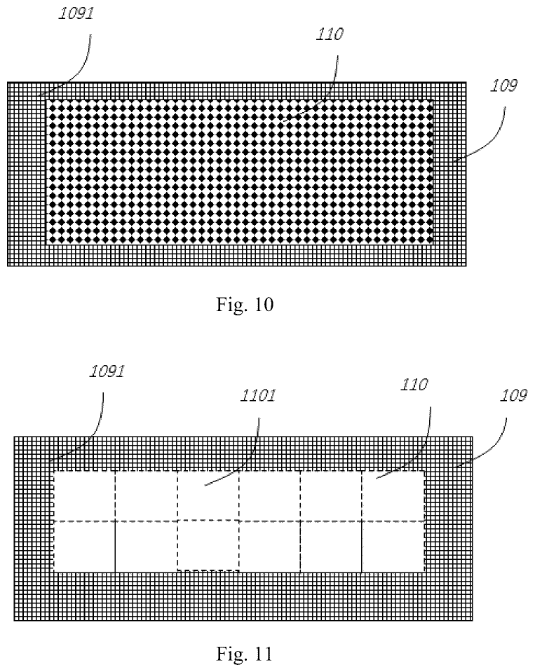

In some embodiments, as shown in FIGS. 8-9, a protective layer 108 covers the back surface 1011 of the second layer. The protective layer is water-permeable but does not absorb the sample, e.g., in a filter mesh form. When the vaginal secretion is in contact with the filter layer 108, large-particle impurities are filtered out, small impurities are then filtered out through the dense mesh pores on the back surface 1011 of the second layer, and the fluid then flows into the middle loose layer for storage. During sampling, the filter layer 108 is torn off, exposing the second layer 1011. Sampling and sample treatment are performed according to the method mentioned above for subsequent testing of analytes, e.g., target nucleic acid testing and tumor marker testing.

In some embodiments, as shown in FIG. 3, a protective layer 201 covers the surface of the second layer. The protective layer may be transparent or non-absorbent, primarily serving to prevent the second layer from contact or contamination. During use, the protective layer 201 is torn off, exposing the back surface of the first layer 101, thereby enabling direct contact with the skin around the vagina to receive or absorb the vaginal secretions.

During specific sampling, areas containing large-particle substances are generally avoided. Sampling can be from the areas free of large-particle substances. Additionally, the sampling may be from the central area of the loose layer 107, with an area of 1 square centimeter or 1.5 square centimeters, or from one area at different locations, followed by combining the samples from these areas for subsequent sample extraction and treatment.

In some embodiments, multiple absorbent materials, for example, absorbent strips, can adhere to a non-absorbent backing; or a piece of absorbent material can adhere, but the material has an easily-torn line structure that can divide the material into different areas. During testing, small areas are torn off directly for sample pretreatment. As shown in FIGS. 11-12, a flexible backing 109 serves as one implementation of a flexible sampling element. The flexible backing 109 is non-absorbent. One or more absorbent areas are disposed on the backing 109. These absorbent areas are made of absorbent materials and can directly cover around the vagina to absorb the secretions in the vagina. These secretions are absorbed by the absorbent materials and retained. For example, an absorbent material layer 110 covers the flexible backing 109. The absorbent material layer can be divided into multiple absorbent areas 1001 and 1102. An adhesive layer is applied to the area around the non-absorbent layer 109 not covered by the absorbent layer 110. In this way, the sanitary pad can directly adhere to the skin around the vagina through the adhesive layer, allowing the absorbent layer 110 to absorb the vaginal secretion. Certainly, a layer of adhesive can be coated on the back surface of the non-absorbent layer 109. The sanitary pad adheres to the underwear through the adhesive layer. After the underwear is worn, the absorbent layer 110 directly covers against the vagina.

In some embodiments, the duration for which the sanitary pad covers the vagina is not strictly limited. It may be over 30 minutes, e.g., 1-12 hours, 5-8 hours, or 8-10 hours. To facilitate sampling and reduce blood contamination, optimal use is recommended after the female menstrual cycle ends. The sanitary pad of the present disclosure can be used to cover the vigina before bedtime at night and removed the following morning. The sanitary pad is couriered to a testing center. Approximately overnight wear while sleeping, the vaginal secretion is absorbed for testing or assays of analytes. Certainly, prior to testing and assays, the sample on the sanitary pad undergoes treatment, such as sample dilution, extraction, or nucleic acid extraction.

In some embodiments, when the sanitary pad has a pH test paper 102, the secretion is absorbed by the pH filter paper 102 disposed between the first layer and the second layer. If the pH of the secretion is abnormal, the absorbent area of the filter paper displays a green color, indicating that pH is positive and the environment is at least not acidic, and may be alkaline or neutral. Certainly, the color-changed area contains the secretion, such that the secretion in this area can be collected for other tests, such as nucleic acid testing, immunoassays, or other biochemical marker tests like testing for oxidase, hydrogen peroxide, leukocyte esterase, sialidase, β-glucuronidase, and acetylglucosaminidase. Therefore, the vaginal environment and potential bacterial or viral infections, for example, the presence or absence of HIV, STDs, and other diseases, are assessed through the test results.

### Testing Method

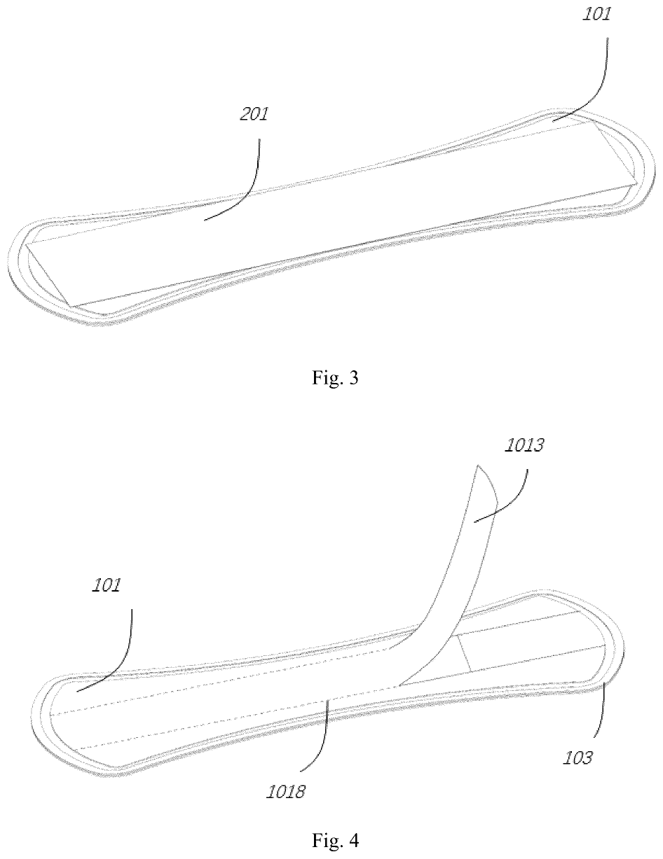

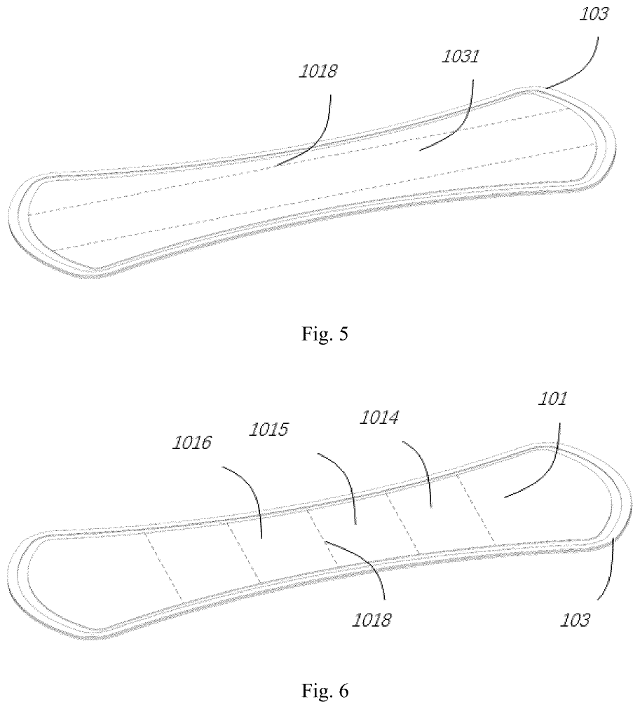

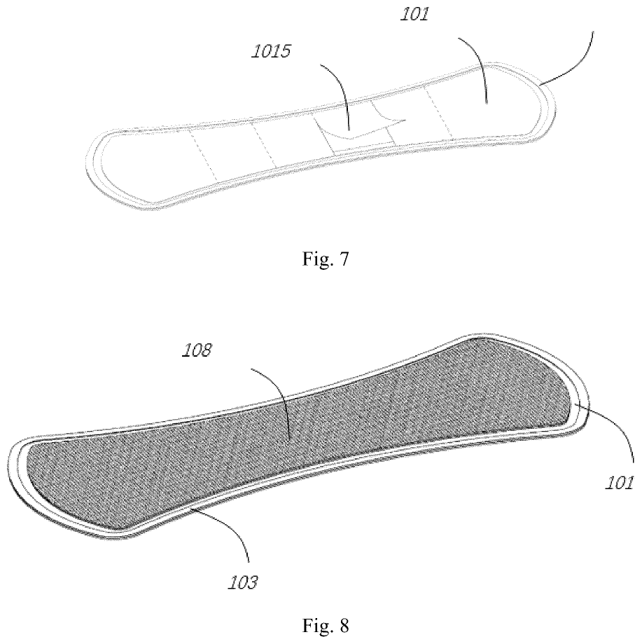

Sanitary pads are a daily-use medical product for women. The present disclosure leverages women's familiarity with sanitary pads, extending their use as tools for collecting vaginal secretions. After collection, the secretions can be tested. For example, as shown in FIGS. 1-3, after collecting the sample at home using the sanitary pad, the user performs self-sampling (e.g., packaging the sanitary pad and sending it to a testing facility). Upon receiving the sanitary pad, the testing facility tests the secretions on it for HPV, STDs, or microbial environment, and then obtains the test result. If the result is positive, therapy is performed according to different conditions. During therapy, at-home self-collection of samples for monitoring can still be performed. For example, as shown in FIG. 4, a flexible sampling element is applied to cover the vaginal surface for approximately 5-8 hours, the vaginal secretion is then collected, and the sample is then obtained from the element used and undergoes pretreatment. The pretreated sample is tested for specific tests such as nucleic acid testing (e.g., for viruses like HPV, HIV, and STDs), and immunoassays (e.g., testing for viral antigens). Certainly, the sample can also be tested for biological testing, e.g., for a pH value.

### DETAILED DESCRIPTION OF THE EMBODIMENTS

How the present disclosure is implemented is illustrated by specific embodiments. However, these specific embodiments do not constitute any limitation on the present disclosure.

### Embodiment 1: Comparison Between Testing of HPV Using Sanitary Pad Sampling and Testing Using Conventional Swab Sampling

Reagents: Buffer AL; Cell Nucleic Acid Extraction Kit (SDK80125); HPV Nucleic Acid Test Kit (fluorescence PCR) (JC80402); Neisseria gonorrhoeae/Chlamydia trachomatis/Ureaplasma urealyticum Nucleic Acid Test Kit (fluorescence PCR) (JB60301N). All the kits were provided by Jiangsu Bioperfectus Technologies Co., Ltd. All the kits are commercially available products.

Instruments: Fully automated nucleic acid extraction instrument; Fluorescence quantitative PCR instrument. Instrument models: Bioperfectus SSNP-3000A; Hongshi SLAN-96 Fully Sutomated Fluorescence Quantitative PCR instrument.

### 1.1 Collection of Vaginal Secretions

The sanitary pad of the present disclosure was used to collect vaginal secretions that were from 50 female patients identified as HPV-positive and positive for three sexually transmitted diseases and subjected to prior cell culture. The patients wore the sanitary pad after bathing in the evening and before bedtime (20:00-22:00) and removed it upon waking (06:00-08:00), with a typical wearing duration of 5-12 hours. The sanitary pads were then sent to the applicant's laboratory for testing and assays. For comparison, 50 cervical swab samples were collected by means of swab sampling (cotton swabs). The average internal control CT value served as a control for comparing exfoliated cell counts in the samples. Specifically, as shown in FIG. 1A, the sample pad was taken out, and the release liner 105 was removed to expose the adhesive for adhering to the underwear. Through the underwear, the dense back surface 1011 of the second layer 101 covered around the vagina to collect secretions. The loose layer 107 of the third layer was located between the first layer 103 and the second layer 101. The loose layer was made of cotton and had an overall thickness of approximately 3-4 mm. A pH test paper was disposed between the first layer 103 and the second layer. In this embodiment, the loose layer 107 adheres to the inner surface 1031 of the first layer 103.

### 1.2. Nucleic Acid Extraction Experiment Procedure

### 1.2.1 Medical Pad Samples

(1) After receiving, the sample was taken out and unfolded. Disinfected scissors were used to cut between the first layer and the second layer. The pH test paper between them was removed. The color of the pH test paper was read to preliminarily judge vaginal inflammation. A pad of approximately 1.5 cm*1.5 cm area was cut off from the central area of the loose layer 107 on the first layer and placed in a 2 mL centrifuge tube.

(2) 800 μL of buffer AL and 20 μL of proteinase K were added to the centrifuge tube, followed by even vortex mixing and instantaneous centrifugation.

(3) The centrifuge tube was placed in a metal bath or water bath for incubation at 56° C. for 20 minutes.

(4) The centrifuge tube was taken out. 300 μL of the incubated liquid was pipetted and added to the reagent plate of SDK80125 Extraction Kit for nucleic acid extraction.

### 1.2.2 Cervical Swab Samples

The swab cotton was inserted in the buffer and shaken well. 300 μL of the sample was taken out and added to the reagent plate of SDK80125 Extraction Kit. Then, 20 μL of proteinase K was added for nucleic acid extraction.

### 1.3 Specific Test Method

### 1.3.1 Preparation of Reaction System:

The amplification reaction system was prepared according to Table 1. A primer-probe mixture and an amplification reaction solution were added to a reagent tube at ratios, and thoroughly mixed to prepare the reaction system, which was instantaneously centrifuged before use.

TABLE 1.1
Reaction System Formulation

| | Addition Volume (μL) per |
|---|---|
| Component | patient |
| Amplification Reaction | 12.5 |
| Solution | |
| Primer-Probe Mixture | 7.5 |
| Total Volume | 20 μL |

Primers used as shown in Table 1.2:

| | |
|---|---|
| HPV Type 16 | SEQ ID NO: 1 |
| | Forward Primer for HPV Type 16 |
| | GAATTCYACTATTTTGGAGGAC (SEQ NO: 1) |
| | SEQ ID NO: 2 |
| | Reverse Primer for HPV Type 16 |
| | TTGACAAGCAATTGCCTGG (SEQ NO: 2) |
| | SEQ ID NO: 3 |
| | Probe for HPV Type 16 |
| | CCAGGAGGCACACTAGAAGATAC (SEQ NO: 3) |
| HPV Type 18 | SEQ ID NO: 4 |
| | Forward Primer for HPV Type 18 |
| | TGACACTGTGCCTCAATCCT (SEQ NO: 4) |
| | SEQ ID NO: 5 |
| | Reverse Primer for HPV Type 18 |
| | ACAACTGGGAGTCAGAGGTAAC (SEQ NO: 5) |
| | SEQ ID NO: 6 |
| | Probe for HPV Type 18 |
| | AGGTGAAGCACGCATACCTGTGCC (SEQ NO: 6) |
| Chlamydia | SEQ ID NO: 7 |
| trachomatis | Forward Primer for Chlamydia trachomatis |
| | GGCTGAAAGAGAAAGAGGTATC (SEQ NO: 7) |
| | SEQ ID NO: 8 |
| | Reverse Primer for Chlamydia trachomatis |
| | GATGAAATCTCTGTGACCTGG (SEQ NO: 8) |
| | SEQ ID NO: 9 |
| | Probe for Chlamydia trachomatis |
| | TTCGAAACTCCAAAATACCACGT (SEQ NO: 9) |
| Neisseria | SEQ ID NO: 10 |
| gonorrhoeae | Forward Primer for Neisseria gonorrhoeae |
| | AGTCAGTGATTTCGGCTCGTT (SEQ NO: 10) |
| | SEQ ID NO: 11 |
| | Reverse Primer for Neisseria gonorrhoeae |
| | GCCTGCCAAGCCGATAAAGGA (SEQ NO: 11) |
| | SEQ ID NO: 12 |
| | Probe for Neisseria gonorrhoeae |
| | AGCTGCCAAACAGCCTTCAG (SEQ NO: 12) |
| Ureaplasma | SEQ ID NO: 13 |
| urealyticum | Forward Primer for Ureaplasma urealyticum |
| | GTGCAAAACCTTACTATGTTGT (SEQ NO: 13) |
| | SEQ ID NO: 14 |
| | Reverse Primer for Ureaplasma urealyticum |
| | TGAACGTCCATATGTTCCAAA (SEQ NO: 14) |
| | SEQ ID NO: 15 |
| | Probe for Ureaplasma urealyticum |
| | TGGGTGTAATTGCTCGTTGTGTAG (SEQ NO: 15) |

The amplification reaction solution contained 3-10 U/test (preferably 8 U/test in this embodiment) of Taq DNA polymerase (purchased from Shanghai ShuoYing Biotechnology Co., Ltd., Model CSMix-005), 3-10 U/test (preferably 8 U/test in this embodiment) of UDG enzyme, 2× Buffer, 0-10 mM (preferably 5 mM in this embodiment) MgCl₂, and 0.5-1 mM (preferably 1 mM in this embodiment) dNTPs (dATP:dTTP:dCTP:dGTP:dUTP=1:1:1:1:1), and prepared using RNase-free water.

### 1.3.2 Amplification Reaction Program

The reagent tubes mentioned above were placed into the SLAN-96 Fluorescent PCR Instrument. The names of samples to be tested, standard substances, and their corresponding concentrations were set, and the amplification testing was then initiated. The multiplex fluorescent PCR reaction conditions are shown in Table 2.

TABLE 2
Amplification Program

| Step | Temperature (° C.) | Time | Cycles |
|---|---|---|---|
| 1 | 50° C. | 2 min | 1 |
| 2 | 95° C. | 10 s | 1 |
| 3 | 95° C. | 5 s | 45 |
| | 58° C.* | 20 s | |

Note:
Fluorescence testing is carried out at 58° C. in Step 3. The fluorescent label varies for each target test.

### II. Data Results

TABLE 3
HPV Test Results

| | | | | | Internal Control | |
|---|---|---|---|---|---|---|
| | Type 16 CT Value | | Type 18 CT Value | | Channel CT Value | |
| | Sanitary | | Sanitary | | Sanitary | |
| | Pad of the | Cervical | Pad of the | Cervical | Pad of the | Cervical |
| Sample | Present | Swab | Present | Swab | Present | Swab |
| Size | Disclosure | Sample | Disclosure | Sample | Disclosure | Sample |
| 50 | with | NoCt: 15; | With | NoCt: 12; | With | With |
| | CT: 48; | with | CT: 45; | with | CT: 50 | CT: 50 |
| | NoCt: 2 | CT: 35 | NoCt: 5 | CT: 38 | | |
| Positive | 96% | 70% | 90% | 76% | 100% | 100% |
| Rate | | | | | | |

TABLE 4
Test Results of 3 STDs (CT/NG/UU)
Neisseria gonorrhoeae/Chlamydia trachomatis/Ureaplasma urealyticum
Nucleic Acid Test Kit (JB60301N)

| | Neisseria | | Chlamydia | | Ureaplasma | | Internal Control | |
|---|---|---|---|---|---|---|---|---|
| | gonorrhoeae | | trachomatis | | urealyticum | | Channel | |
| | CT value | | CT value | | CT value | | CT Value | |
| | Sanitary | | Sanitary | | Sanitary | | Sanitary | |
| | Pad of the | Cervical | Pad of the | Cervical | Pad of the | Cervical | Pad of the | Cervical |
| Sample | Present | Swab | Present | Swab | Present | Swab | Present | Swab |
| Size | Disclosure | Sample | Disclosure | Sample | Disclosure | Sample | Disclosure | Sample |
| 50 | CT: 22 | CT: 15 | CT: 25 | CT: 0 | CT: 25 | CT: 20\ | CT: 50 | CT: 50 |
| | Noct: 28 | NO ct: 35 | No: 25 | No: 50 | No CT: 5 | NoCT: 30 | | |
| Positive | 44% | 30% | 50% | 0% | 50% | 40% | 100% | 100% |
| Rate | | | | | | | | |

Note:
samples with a CT value less than 30.0 are considered positive; samples with no CT value are considered negative.

It can be seen from the above that for the 50 positive samples, the use of the sanitary pad of the present disclosure for sample collection can achieve a relatively high positive test rate. In contrast, while cervical swab samples demonstrate a certain positive rate, it is significantly lower. This may be likely because sample collection using the medical pad of the present disclosure can collect a larger sample volume, and the collection time is longer such that more reliable test results can be obtained; furthermore, it captures a higher proportion of infected cells, thereby contributing to the higher positive test rate.

### Embodiment 2: Comparison Between Positive Test Rates of Different Sampling Areas of the Sanitary Pad of the Present Disclosure

The specific procedure was shown in that in Embodiment 1. For testing different sampling positions on the sanitary pad, the impact on the positivity rate of samples obtained from the optimal sampling position was compared. The following sampling positions were used: (1) The dense surface of the second layer (with the loose layer removed); the loose layer between the second layer and the first layer; and the dense surface of the first layer. Sampling was performed in the central area of approximately 1.5 cm*1.5 cm for all positions. The HPV test results are shown below:

TABLE 5
HPV Test Results

| | Type 16 CT Value | | | Type 18 CT Value | | |
|---|---|---|---|---|---|---|
| | Dense | | Dense | Dense | | Dense |
| | Surface | | Surface | Surface | | Surface |
| | of Second | Loose | of First | of Second | Loose | of First |
| | Layer | Layer | Layer | Layer | Layer | Layer |
| Positive | 80% | 96% | 76% | 82% | 90% | 65% |
| Rate | | | | | | |

It can be seen from the results that sampling from the intermediate loose layer obtained a higher positivity rate. Sampling from other surfaces had a lower positivity rate likely due to more interfering substances and simple nucleic acid extraction. Additionally, due to variations in sanitary pad-wearing methods, the second layer in direct contact with the vagina showed a higher positive rate than the first layer (farther from the vagina). This may be because less sample transfer occurred to the second layer, affecting the positive rate.

### Embodiment 3: Impact of Different Wearing Methods on Positive Rates of Test Samples

Compared with Embodiment 1, the adhesive layer directly covered the skin around the vagina. Adhesive 104 was applied to the dense surface of the first layer, allowing vaginal secretions to be first in contact with the adhesive layer 104, then enter the loose layer 107, and then enter the dense layer of second layer 101. Certainly, the vaginal secretions can be in contact with the intermediate pH test paper. Sampling was performed in the central area of the intermediate loose layer.

TABLE 6
HPV Test Results

| | Type 16 CT Value | | | Type 18 CT Value | | |
|---|---|---|---|---|---|---|
| | Dense | | Dense | Dense | | Dense |
| | Surface | | Surface | Surface | | Surface |
| | of Second | Loose | of First | of Second | Loose | of First |
| | Layer | Layer | Layer | Layer | Layer | Layer |
| Positive | 79% | 92% | 76% | 80% | 89% | 64% |
| Rate | | | | | | |

It can be seen from the above that the positivity rate achieved by direct contact of the adhesive layer with the external vaginal surface was comparable to that achieved by direct contact of the second layer with the external vaginal surface. Specific positive rates differed from those in Table 5, and this is likely due to sample error caused by different nucleic acid amplification methods. However, the overall positive rates were relatively consistent.

### Embodiment 4: Impact of Absence of a Dense Layer on Test Sample Results

Compared with Embodiment 1, the second layer had no dense layer. Instead, the loose layer 107 was in direct contact with the vaginal surface to collect secretions, with sampling also performed in the central area. The HPV test results are shown in Table 7 below.

| | Type 16 CT Value | | | Type 18 CT Value | | |
|---|---|---|---|---|---|---|
| | | | Dense | | | Dense |
| | | Loose | Surface of | | Loose | Surface of |
| | / | Layer | First Layer | / | Layer | First Layer |
| Positive | / | 85% | 76% | / | 80% | 63% |
| Rate | | | | | | |

It can be seen from the results from Table 7 that sampling by direct contact of the loose layer with the vagina resulted in reduced positive rates. This may be due to the absence of the dense layer's filtration for large-particle substances in secretions; using simple nucleic acid extraction resulted in more impurities in nucleic acid solutions, thereby affecting the entire nucleic acid amplification system and lowering the positive rate. However, the samples filtered through the loose layer and flowing to the first layer for nucleic acid testing showed no significant change in the positive rate.

### Embodiment 5: Testing of Vaginal Microbial Species Using Samples Collected with the Sanitary Pad of the Present Disclosure

This embodiment refers to the applicant's published invention patent, Application No. 202510442876X, filed on Apr. 10, 2025, which discloses a test for 32 microbial species. It aims to evaluate the comprehensiveness of samples collected using the sanitary pad of the present disclosure, with cervical swabs and vaginal swabs serving as controls. The specific nucleic acid reagents, primers, and grouping methods were all the specific sequences and combinations from Embodiment 1 of the published patent. Meanwhile, the testing method for species and types was the method in Embodiment 2. To verify the experiment, cell culture of secretions was used to verify the authenticity and consistency of different sampling methods (samples were collected from 10 female patients with vaginal inflammation confirmed by pH testing). Sampling methods included the sanitary pad of the present disclosure (worn for 5-8 hours), traditional cervical swabs (1-2 minutes, cotton), and vaginal swabs (30 seconds to 1 minute, cotton). Each method was used only once. The sanitary pad of the present disclosure was worn before bedtime at night and removed upon waking in the morning, then returned to the laboratory. The wearing time was 5-8 hours. For test result counting, only positive results were counted; the absence of a result was recorded as “No result”.

The specific results are as follows:

| | | Sanitary | | | |
|---|---|---|---|---|---|
| | | Pad of the | | | |
| | Microorganism | Present | Vaginal | Cervical | Cell |
| No. | Tested | Disclosure | Swab | Swab | Culture |
| 1 | Lactobacillus | 8 Positive | No result | 2 Positive | 8 Positive |
| | crispatus | | | | |
| 2 | Lactobacillus | 2 Positive | 1 Positive | 1 Positive | 3 Positive |
| | jensenii | | | | |
| 3 | Lactobacillus | 5 Positive | 1 Positive | 1 Positive | 5 Positive |
| | gasseri | | | | |
| 4 | Lactobacillus | 4 Positive | 1 Positive | 1 Positive | 5 Positive |
| | iners | | | | |
| 5 | Atopobium | 8 Positive | No result | No result | 8 Positive |
| | vaginae | | | | |
| 6 | Prevotella | 7 Positive/ | 7 Positive/ | 7 Positive/ | 7 Positive/ |
| 7 | Mycoplasma | 8 Positive/ | No result | No result | 8 Positive/ |
| | genitalium | | | | |
| 8 | Mycoplasma | 8 Positive/ | 8 Positive/ | 8 Positive/ | 8 Positive/ |
| | hominis | | | | |
| 9 | Gardnerella | 7 Positive/ | No result | No result | 8 Positive/ |
| | vaginalis | | | | |
| 10 | Bacteroides | 6 Positive | 6 Positive | 6 Positive | 6 Positive |
| 11 | Mobiluncus | 4 Positive/ | 4 Positive/ | 4 Positive/ | 4 Positive/ |
| 12 | Herpes Simplex | 4 Positive/ | No result | No result | 5 Positive/ |
| | Virus Type 2 | | | | |
| 13 | Candida | 4 Positive/ | 1 Positive/ | 3 Positive/ | 4 Positive/ |
| | albicans | | | | |
| 14 | Candida | 3 Positive/ | 3 Positive/ | 3 Positive/ | 3 Positive/ |
| 15 | Escherichia coli | 7 Positive/ | No result | No result | 8 Positive/ |
| 16 | Staphylococcus | 8 Positive/ | 8 Positive/ | 8 Positive/ | 8 Positive/ |
| | aureus | | | | |
| 17 | Neisseria | 8 Positive/ | No result | No result | 8 Positive/ |
| | gonorrhoeae | | | | |
| 18 | Chlamydia | 7 Positive/ | 2 Positive/ | 4 Positive/ | 8 Positive/ |
| | trachomatis | | | | |
| 19 | Ureaplasma | 8 Positive/ | No result | No result | 8 Positive/ |
| | urealyticum | | | | |
| 20 | Trichomonas | 6 Positive/ | 6 Positive/ | 6 Positive/ | 8 Positive/ |
| | vaginalis | | | | |
| 21 | HPV Type 16 | 10 | No result | 5 Positive/ | 10 |
| | | Positive/ | | | Positive/ |
| 22 | HPV Type 18 | 7 Positive/ | 7 Positive/ | 7 Positive/ | 10 |
| | | | | | Positive/ |
| 23 | HPV Type 31 | 5 Positive/ | 2 Positive/ | 2 Positive/ | 5 Positive/ |
| 24 | HPV Type 35 | 2 Positive/ | 1 Positive/ | No result | 2 Positive/ |
| 25 | HPV Type 53 | 2 Positive/ | No result | 2 Positive/ | 2 Positive/ |
| 26 | HPV Types 56 | 3 Positive/ | 3 Positive/ | 3 Positive/ | 3 Positive/ |
| | and 66 | | | | |
| 27 | HPV Types 39 | 4 Positive/ | 3 Positive/ | No result | 6 Positive/ |
| | and 68 | | | | |
| 28 | HPV Types 33 | 4 Positive/ | No result | 2 Positive/ | 4 Positive/ |
| | and 58 | | | | |
| 29 | HPV Types 51 | 3 Positive/ | 3 Positive/ | 3 Positive/ | 3 Positive/ |
| | and 82 | | | | |
| 30 | HPV Type 26 | 5 Positive/ | 5 Positive/ | 5 Positive/ | 5 Positive/ |
| 31 | HPV Type 73 | 6 Positive/ | 4 Positive/ | 2 Positive/ | 10 |
| | | | | | Positive/ |
| 32 | HPV Type 45 | 8 Positive/ | 1 Positive/ | 1 Positive/ | 8 Positive/ |
| 33 | HPV Type 52 | 8 Positive/ | 8 Positive/ | 8 Positive/ | 8 Positive/ |
| 34 | HPV Type 59 | 8 Positive/ | 4 Positive/ | No result | 8 Positive/ |
| 35 | Group B | 7 Positive/ | 7 Positive/ | 7 Positive/ | 10 |
| | Streptococcus | | | | Positive/ |
| 36 | Megasphaera | 10 | No result | No result | 10 |
| | | Positive/ | | | Positive/ |
| 37 | BVAB2 | 6 Positive/ | 6 Positive/ | 6 Positive/ | 6 Positive/ |
| 38 | Treponema | 7 Positive/ | 7 Positive/ | 7 Positive/ | 7 Positive/ |
| | pallidum | | | | |
| 39 | Herpes Simplex | 6 Positive/ | No result | 6 Positive/ | 6 Positive/ |
| | Virus Type 1 | | | | |
| 40 | Streptococcus | 8 Positive/ | 8 Positive/ | 8 Positive/ | 8 Positive/ |
| | anginosus | | | | |
| 41 | Enterococcus | 9 Positive/ | No result | 9 Positive/ | 10 |
| | faecalis | | | | Positive/ |
| 42 | Klebsiella | 8 Positive/ | 8 Positive/ | No result | 8 Positive/ |
| | pneumoniae | | | | |
| 43 | Group A | 9 Positive/ | 9 Positive/ | 9 Positive/ | 10 |
| | Streptococcus | | | | Positive/ |

It can be seen from the test results that sampling using the sanitary pad of the present disclosure can at least comprehensively capture samples and most of the 43 microbial species can be effectively tested, aligning closely with the cell experiment despite minor discrepancies. However, for sampling using vaginal and cervical swabs, some cannot obtain test results, indicating a high rate of missed detection. This causes inaccuracy in identifying the specific microbial community type, which may directly impact subsequent therapeutic measures. For example, infections caused by different types such as viruses and bacteria require different therapeutic measures.

All patents and publications mentioned in the specification of the present disclosure indicate that these are disclosed techniques in the art and can be used by the present disclosure. All patents and publications cited herein are likewise listed in the references as if each publication is specifically and separately referenced. The present disclosure described herein may be implemented in the absence of any one or more elements, and one or more limitations, which are not specifically stated herein. For example, the terms “including”, “essentially consisting of”, and “consisting of” in each embodiment herein may be replaced by the rest 2 terms. The term “a/an” herein merely means “one”, but does not exclude including 2 or more instead of including only one. The terms and expressions employed herein are descriptive and are not limited thereto, and there is no intention herein to indicate that the terms and interpretations described herein exclude any equivalent features, but it can be noted that any appropriate changes or modifications can be made within the scope of the present disclosure and claims. It can be understood that the embodiments described in the present disclosure are preferred embodiments and features, and any person having ordinary skill in the art can make some modifications and changes based on the essence of the description of the present disclosure. These modifications and changes are also considered to be within the scope of the present disclosure and the scope limited by the independent claims and the dependent claims.

1. A system for testing an analyte in a female vaginal secretion, comprising:
a collecting element for collecting a female vaginal secretion, wherein the collecting element covers an external surface of a vagina instead of entering into the vagina, which is configured to absorb the vaginal secretion; and
a testing element for testing an analyte in the collected the vaginal secretion or a property of the the vaginal secretion, wherein a test result obtained by the testing element is set to assess if the the vagina is a health status.

2. The system according to claim 1, wherein the collecting element comprises a sheet-like absorbent material that covers the external surface of the vagina and is used for absorbing the vaginal secretion from the vagina.

3. The system according to claim 2, wherein the sheet-like absorbent material comprises a feminine sanitary pad or a urine absorbent pad, and the feminine sanitary pad is configured to cover the surface of the female vagina for a specific time.

4. The system according to claim 3, wherein the time is 2-20 hours, and the time for covering is before bedtime at night.

5. The system according to claim 4, wherein the feminine sanitary pad is provided with different areas, and one area is capable of being detachably removed from the feminine sanitary pad to be used as test samples.

6. The system according to claim 5, wherein the different areas of the feminine sanitary pad are capable of being directly cut off to obtain partial samples.

7. The system according to claim 6, wherein the feminine sanitary pad comprises a first layer and a second layer, a loose layer for absorbing a sample is disposed between the first layer and the second layer, and the first layer and the second layer are dense absorbent layers.

8. The system according to claim 7, wherein the loose layer is capable of being pulled out or separated from between the second layer and the first layer.

9. The system according to claim 7, wherein the first layer has a front surface and a back surface, the second layer also has a front surface and a back surface, and the loose layer is capable of being fixed on the front surface of the first layer or on the front surface of the second layer.

10. The system according to claim 9, wherein the back surface of the first layer has an adhesive layer for adhering the sanitary pad to an underwear or to a skin around the vagina.

11. The system according to claim 10, wherein the testing element comprises a nucleic acid testing element, an immunoassay element, or a biochemical testing element.

12. The system according to claim 11, wherein the nucleic acid testing element is used for testing a quantity or presence or absence of a target nucleic acid in the female vaginal secretion, and preferably, the target nucleic acid comprises a virus, a bacterium, or a fungus, more preferably, the virus.

13. The system according to claim 12, wherein the virus is human papillomavirus (HPV) or a sexually transmitted disease (STD).

14. The system according to claim 13, wherein the STD comprises syphilis, condyloma acuminata, acquired immune deficiency syndrome, or gonorrhea.

15. The system according to claim 14, wherein the nucleic acid testing element comprises necessary reagents for polymerase chain reaction (PCR), isothermal nucleic acid amplification.

16. The system according to claim 13, wherein the system further comprising a nucleic acid extraction reagent for extracting the nucleic acid from the loose layer of the sanitary pad.

17. The system according to claim 11, wherein biochemical testing or immunoassay is for testing a viral antigen, or oxidase, hydrogen peroxide, leukocyte esterase, sialidase, β-glucuronidase, acetylglucosaminidase, a pH value, or a tumor marker in the analyte.

18. A method for testing an analyte in a female vaginal secretion, comprising: covering an external surface of a vagina with a sheet-like element comprising an absorbent material for a specific time for absorbing a vaginal secretion from the vagina; and taking off the sheet-like element and then removing a part of the absorbent material comprising the secretion from the sheet-like element for testing an analyte in the female vaginal secretion.

19. The method according to claim 18, wherein the sheet-like element is a feminine sanitary pad.

20. The method according to claim 18, wherein the removing comprises cutting off the sheet-like absorbent material from the sheet-like element or tearing off the sheet-like absorbent material from the sheet-like element.

21. The method according to claim 18, wherein the time for covering comprises 2-20 hours, preferably 5-8 hours.

22. The method according to claim 21, wherein the covering is self-applied by a patient on her own external vaginal surface; and the removing a part of the absorbent material comprising the secretion is performed by laboratory personnel.

23. The method according to claim 19, wherein the feminine sanitary pad is provided with different areas, and one of the areas is capable of being detachably removed from the feminine sanitary pad to be used as a test sample.

24. The method according to claim 23, wherein the different areas of the feminine sanitary pad are capable of being directly cut off to obtain partial samples.

25. The method according to claim 19, wherein the sanitary pad comprises a first layer and a second layer, a loose layer for absorbing a sample is disposed between the first layer and the second layer, and the first layer and the second layer are dense absorbent layers.

26. The method according to claim 25, wherein the first layer has a front surface and a back surface, the second layer also has a front surface and a back surface, and the loose layer is capable of being fixed on the front surface of the first layer or on the front surface of the second layer.

27. The method according to claim 26, wherein the back surface of the first layer has an adhesive layer for adhering the sanitary pad to an underwear or to a skin around the vagina.
